(19) United States
(12) Patent Application Publication
(10) Pub. No.: US 2025/0346541 A1
(43) Pub. Date: Nov. 13, 2025

(54) PROCESS FOR UTILIZING CARBON OXIDES IN A FLUE GAS STREAM

(71) Applicant: UOP LLC, Des Plaines, IL (US)

(72) Inventors: Ernest J. Boehm, Hanover Park, IL (US); Richard A. Johnson, II, Algonquin, IL (US); Michael J. Tobin, Elmhurst, IL (US)

(21) Appl. No.: 18/659,686

(22) Filed: May 9, 2024

(51) Int. Cl. C07C 1/02; B01D 53/62

(52) U.S. Cl. CPC C07C 1/02; B01D 53/62; B01D 2251/102; B01D 2257/502; B01D 2258/0283

(57) ABSTRACT

A process of separating carbon oxides from a flue gas stream is disclosed. The process comprises separating a flue gas stream into a first flue gas stream and a second flue gas stream. The first flue gas stream is combusted with an oxygen stream in a boiler to provide a carbon dioxide rich flue gas stream. A carbon dioxide recycle stream is taken from the carbon dioxide rich flue gas stream. The second flue gas stream is recycled to an oxygenate production unit. The carbon dioxide recycle stream is recycled to the boiler or to a regenerator or both.

### FIELD

The field is related to a process of separating carbon oxides from a flue gas stream. Particularly, the field relates to a process of separating carbon oxides from a flue gas stream and taking a recycle stream and a feed stream from the separated carbon oxides.

### BACKGROUND

Carbon dioxide is a so-called greenhouse gas which concentration many desire to suppress in the atmosphere. Carbon oxides may be converted to oxygenates such as methanol or dimethyl ether. Molecular sieves such as microporous crystalline zeolite and non-zeolitic catalysts, particularly silicoaluminophosphates (SAPO), are known to promote the conversion of oxygenates to hydrocarbon mixtures, particularly hydrocarbon mixtures composed largely of light olefins. The highly efficient methanol to olefin (MTO) process may convert oxygenates to light olefins which had been typically considered for plastics production and recently for producing sustainable aviation fuel (SAF). Light olefins produced from the MTO process are highly concentrated in ethylene and propylene.

Alternative processes are also used for light olefins production. In one approach, hydrocarbon oxygenates and more specifically methanol or dimethyl ether are used as an alternative feedstock for producing light olefin products. Once the oxygenates are formed, the process includes catalytically converting the oxygenates, such as methanol, into the desired light olefin products in the MTO process. In the MTO process, carbonaceous material, i.e., coke, is deposited on the catalyst as it moves through the reaction zone. The carbonaceous material is removed from the catalyst by oxidative regeneration in a regeneration zone wherein a moving bed of the catalyst particles withdrawn from the reaction zone is contacted with an oxygen-containing gas stream at sufficient temperature and oxygen concentration to allow the desired amount of the carbonaceous materials to be removed by combustion from the catalyst. In some cases, it is advantageous to regenerate the catalyst only partially by removing from about 30 to 80 wt-% of the carbonaceous material. During this regeneration there is incomplete combustion of coke resulting in a mixture of water, carbon dioxide and carbon monoxide. The ratio of net carbon dioxide to carbon monoxide produced during regeneration may range from about 1.0 to about 10.0. This will result in a carbon monoxide concentration in the flue gas between about 0 mol % and about 8 mol %.

Light olefin oligomerization is a process that can perform the conversion of C2 through C6 olefins into more desirable products. More specifically, it can convert C2 through C6 olefins into liquid fuels, including naphtha, jet fuel, and diesel range products.

Jet fuel is one of the few petroleum fuels that cannot be replaced easily by electrical motor systems because a high energy output is required to fuel planes which cannot be supplied with electric motors. Large incentives are currently available for green or renewable jet fuel in certain regions.

Conventional catalyst regenerators typically include a vessel having a coked catalyst inlet, a regenerated catalyst outlet and a combustion gas distributor for supplying air or other oxygen containing gas to the bed of catalyst that resides in the vessel. Cyclone separators remove catalyst entrained in the flue gas before the gas exits the regenerator.

Flue gas formed by burning the coke in the regenerator is treated for removal of particulates and conversion of carbon monoxide, after which the flue gas is normally discharged into the atmosphere. Further, incomplete combustion to carbon monoxide can result from poor fluidization or aeration of the coked catalyst in the regenerator or poor distribution of coked catalyst into the regenerator. Generally, the flue gas exiting the regenerator contains carbon monoxide, carbon dioxide, nitrogen and water, along with smaller amounts of other species. Flue gas treatment methods are effective, but the capital and operating costs are high.

Environmental concerns over greenhouse gas emissions have led to an increasing emphasis on separating the greenhouse gases before releasing the flue gases into atmosphere. Carbon dioxide is the most significant long-lived greenhouse gas in earth's atmosphere. Carbon dioxide capture from flue gases is expensive, both from a capital expenditure and operational utility cost standpoint. For fluidized catalytic processes, air is used for regenerating the spent catalyst. As a result of this operation, the carbon dioxide in the flue gas has a lower concentration in contrast to the larger concentrations of other impurities. The presence of other gaseous impurities increases capital expenditure because larger equipment must be employed to handle a larger volume of flue gas.

In an MTO unit, carbon dioxide may be captured from the MTO regenerator flue gas using a solvent process, which requires a great deal of additional equipment and energy to regenerate the solvent. Larger operational utility costs are necessary to remove the impurities by solvent due to higher solvent circulation rates and solvent regeneration duties. Apart from this, the flue gas requires extensive pretreatment to meet stringent specifications necessary to avoid high solvent degradation rates.

With decades of research and recent changes to government regulations, there is a great need to find ways to reduce carbon intensity of products such as petrochemicals, biofuels, or efuels from an MTO unit.

Therefore, there is a need for improved processes for treating flue gas containing carbon dioxide. Also, there is a need for a process and an apparatus which reduces capital expenditure and operational utility cost for capturing carbon dioxide, while improving energy efficiency and energy recovery.

### SUMMARY

The present disclosure provides a process for separating carbon oxides from a flue gas stream is disclosed. The process comprises separating a flue gas stream into a first flue gas stream and a second flue gas stream. The first flue gas stream is combusted with an oxygen stream in a boiler to provide a carbon dioxide rich flue gas stream. A carbon dioxide recycle stream is taken from the carbon dioxide rich flue gas stream. The second flue gas stream is recycled to an oxygenate production unit. The carbon dioxide recycle stream is recycled to the boiler or to a regenerator or both.

### BRIEF DESCRIPTION OF THE DRAWINGS

The various embodiments will hereinafter be described in conjunction with the following FIGURE, wherein like numerals denote like elements.

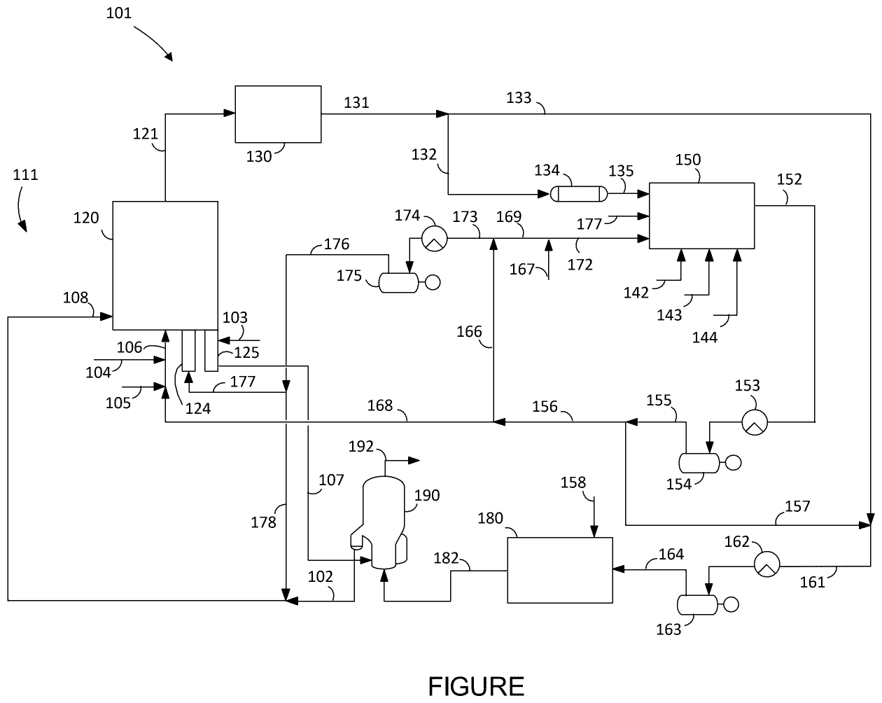

The FIG. 1s a schematic diagram of a process of separating carbon oxides from a flue gas stream in accordance with an embodiment of the present disclosure.

### DEFINITIONS

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” or “directly” means that flow from the upstream component enters the downstream component without passing through a fractionation or conversion unit to undergo a compositional change due to physical fractionation or chemical conversion.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripper columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “a component-rich stream” means that the rich stream coming out of a vessel has a greater concentration of the component than the feed to the vessel.

As used herein, the term “rich” means greater than 50%, suitably greater than 75% and preferably greater than 90%.

### DETAILED DESCRIPTION

Oxygenate conversion process such as MTO produce a flue gas stream. The flue gas stream in the absence of a suitable alternate use, is usually vented to the atmosphere. The present process of separating carbon oxides reduces this emission by providing a solution to utilize the flue gas within the process and also charge it to another process as feed. The present process provides using the flue gas stream comprising carbon oxides to provide a synthetic air stream. Typically, the atmospheric air is used in a catalyst regenerator and/or a CO Boiler of the MTO process. The incoming air to the regenerator provides combustion oxygen and volumetric flow for maintaining fluidization velocity in the MTO regenerator, lift riser, and the regenerator catalyst coolers. The incoming air to the CO boiler provides combustion oxygen and inert gas flow to help remove excess heat generated by combustion to help maintain the internal temperature of the flue gas CO boiler.

The present process comprises capturing carbon oxides by sending the carbon oxide rich flue gas stream to an MTO unit. The carbon oxide rich flue gas stream is converted to an oxygenate such as methanol in an oxygenate production unit. The current process comprises capturing the carbon oxides as methanol. The methanol can be used in applications to make olefins and fuels. The present process captures carbon oxides generated from combusting coke on the MTO catalyst and combusting in the CO Boiler carbon monoxide, a heavy oxygenates waste stream, and/or a small amount of a fuel gas. The MTO process continuously generates coke on the catalyst, and there is a continuous stream of catalyst flowing to the regenerator that emits carbon dioxide from coke combustion. The regenerator may also be operated in partial burn operational mode, so carbon monoxide may be generated which is not fully combusted inside the regenerator. Carbon monoxide is a preferable reactant for the production of methanol.

The present process splits the carbon monoxide rich flue gas into two streams, one going to the regenerator and/or the CO boiler and the other to be fed directly to an oxygenate production unit. The present process also sends a heavy oxygenates stream to the CO boiler. The heavy oxygenates stream burned in the CO boiler may have been recovered from the MTO product stream. The heavy oxygenates stream is sent to the CO boiler to efficiently dispose of it and generate steam. The fuel gas is provided to the CO boiler at a small flow rate to provide fuel for the burner pilot but also in a large enough flow rate as needed to help regulate the internal temperature of the CO boiler. The fuel gas also must be available in the event the supply of the heavy oxygenates stream to the CO boiler is stopped.

Synthetic air may be defined as a mixture of carbon oxides, water, and oxygen. The use of carbon dioxide as one of the components of the synthetic air in the present process is also attractive because nitrogen is detrimental to the methanol synthesis reaction which makes methanol from reacting hydrogen and carbon dioxide. Replacing the atmospheric air with synthetic air as in the present process allows a carbon oxides rich stream with a reduced nitrogen content to be generated that can be used to capture and utilize carbon oxides. If fresh carbon dioxide is made up at both the CO boiler and the lift gas and fluffing gas inlets of the MTO unit, this carbon dioxide effectively passes through the MTO unit and is sent to the oxygenate production unit.

The present process also provides reducing the make-up carbon dioxide to the MTO unit and only compressing necessary make up carbon dioxide and combustion product carbon dioxide to reduce compression energy cost. The process achieves this reduction by recycling low pressure CO boiler flue gas as lift and fluffing gas to the MTO regenerator or blending with pure oxygen to make synthetic air for combustion and heat moderation in the CO boiler.

The present process also reduces the compression energy cost for compressing the carbon dioxide. The CO boiler flue gas stream is used for providing a synthetic air stream to the CO boiler and to the regenerator. By recycling the CO boiler flue gas stream, the present process reduces the compression energy cost for compressing the make-up carbon dioxide.

Referring to FIGURE, a process 101 of separating carbon oxides from a flue gas stream is shown. The process 101 comprises an MTO unit 111 and a boiler 150. The MTO unit 111 comprises a regenerator 120, an oxygenate production unit 180, and an oxygenate conversion unit comprising an MTO reactor 190. As shown, a spent catalyst stream in line 102 is passed to the regenerator 120 to burn coke from the spent catalyst and provide regenerated catalyst. In an embodiment, the spent catalyst stream in line 102 is combined with a fluidization gas stream in line 178 as described later in detail to provide a spent catalyst charge stream in line 108. The spent catalyst charge stream in line 108 is passed to the regenerator 120. The regenerator 120 can be a partial burn regenerator or a complete burn regenerator. In an exemplary embodiment, the regenerator 120 is a partial burn regenerator. A synthetic air stream comprising a carbon dioxide rich oxidation stream in line 106 is passed to the regenerator 120 to burn coke from the spent catalyst and produce a flue gas stream in line 121. The flue gas stream in line 121 comprises carbon oxides, catalyst fines, water and some inert material. Particularly, the flue gas stream in line 121 comprises unconverted carbon monoxide. The unconverted carbon monoxide in the flue gas stream in line 121 can be combusted to carbon dioxide in a boiler 150. In an exemplary embodiment, the boiler 150 is a CO boiler. The present process comprises separating the flue gas stream in line 121 to provide a methanol feed stream for the MTO unit 111 in line 182 and the carbon dioxide rich oxidation stream in line 106.

In an exemplary embodiment, the regenerator 120 is a regenerator of the MTO process. The MTO process continually generates coke on the catalyst, and there is a continuous stream of catalyst flowing in and out of the MTO regenerator 120 that generates carbon dioxide by coke combustion in the regenerator. Typically, the MTO regenerator 120 runs in a partial burn operational mode. In partial burn operation, combustion of the coke inside the regenerator produces carbon monoxide which is not fully combusted inside the regenerator along with carbon dioxide. Carbon monoxide is a preferable reactant for the production of methanol. It is proposed that the unburned carbon monoxide from the MTO regenerator 120 can be suitably used as a feed for the MTO unit 111, particularly for oxygenate production unit 180 to produce methanol. The present process comprises separating the regenerator flue gas stream in line 121 into a first flue gas stream and a second flue gas stream before passing the flue gas stream to the CO boiler 150 and charging the second flue gas stream to the oxygenate production unit 180.

In an aspect, the flue gas stream in line 121 may be passed to a filter section 130 to remove particulates including any catalyst fines from the flue gas stream in line 121 to provide a filtered flue gas stream in line 131. The filter section 130 may comprise a bag filter or an electrostatic precipitator. In one embodiment of the process, the filter section 130 is a bag filter. The bag filter may operate at atmospheric pressure. In an alternative embodiment, the filter 130 may be a high pressure filter designed to operate at nearly the same pressure as the regenerator 120. The advantage of a high-pressure filter is to maintain high pressure and avoid having to recompress the stream in downstream compressors thus reducing compression power required. The filtered material from the filter section 130 may include catalyst fines which may be removed from the filter section 130. A filtered flue gas stream is taken in line 131 and passed to the CO boiler 150.

In an aspect, the operating pressure of the regenerator 120 may be between about 70 kPa(g) (10 psig) and about 350 kPa(g) (50 psig), depending on the processing objectives of the MTO reactor 190. The operating pressure of the filter 130 may be between about 70 kPa(g) (10 psig) and 350 kPa(g) (50 psig) or between about 10 kPa(g) (1.5 psig) and atmospheric pressure, depending on the design constraints of the filter type. This difference in pressure potentially represents a significant amount of energy.

In an embodiment, the filtered flue gas stream taken in line 131 may be separated into a first flue gas stream in line 132 and a second flue gas stream in line 133. In an exemplary embodiment, the second flue gas stream in line 133 may comprise about 20 wt % to about 80 wt % of the flue gas stream in line 121. In another exemplary embodiment, the second flue gas stream in line 133 may comprise about 30 wt % to about 70 wt % of the flue gas stream in line 121. In an aspect, at least 50 wt % of the flue gas stream in line 121 bypasses the CO boiler 150 and is taken in the second flue gas stream in line 133. Indeed, the portion of the flue gas stream in line 121 taken in the second flue gas stream in line 133 should be maximized because it will preserve carbon monoxide in the flue gas stream which is a better reactant for methanol synthesis in the oxygenate production unit 180. The flow rate of the first flue gas stream taken to the CO boiler 150 is only that necessary to operate the CO boiler because it produces a flue gas stream in line 152 which is depleted of carbon monoxide and may be characterized as a carbon dioxide-rich flue gas stream. The carbon dioxide-rich flue gas stream from the CO boiler 150 in line 152 provides a plurality of process streams which may be supplied to one or more of the regenerator 120, the CO boiler 150, a catalyst cooler 124 and the catalyst line 108 as lift gas for spent catalyst as described later in detail. The remainder of the flue gas stream in the second flue gas line 133 is a well-suited composition for the oxygenate production unit 180 due to its higher concentration of carbon monoxide.

The first flue gas stream in line 132 is passed to the CO boiler 150. In an aspect, the first flue gas stream in line 132 may be expanded before passing it to the CO boiler 150. In an exemplary embodiment, the first flue gas stream in line 132 may be passed to an orifice chamber 134 to provide an expanded first flue gas stream in line 135. An orifice chamber is a vertical or horizontal chamber containing a series of perforated plates, designed to maintain a reasonable pressure drop across the control valve. The chamber and the plates, are generally metallic. However, the orifice chamber may be made of ceramic, at least as regards its internal plates. In an alternate embodiment, the first flue gas stream in line 132 may be passed through a valve to provide an expanded first flue gas stream in line 135. The expanded first flue gas stream in line 135 is passed to the CO boiler 150.

The unconverted carbon monoxide in the first flue gas stream can be combusted to carbon dioxide in a CO boiler 150 to produce high-pressure steam. The first flue gas stream may be combusted with an oxygen stream in the CO boiler 150 to combust the carbon monoxide and provide a carbon dioxide rich flue gas stream in line 152. The carbon dioxide rich flue gas stream in line 152 may be passed to the oxygenate production unit 180 of the MTO unit 111. Instead of atmospheric air, the use of synthetic air in this case particularly is attractive because nitrogen is detrimental to the methanol synthesis reaction in the oxygenate production unit 180. Nitrogen has to be removed from the carbon dioxide rich flue gas stream in line 152 before it is passed to the oxygenate production unit 180. In removing the nitrogen for the oxygenate production unit 180, some of the carbon dioxide is also removed with the nitrogen requiring more carbon dioxide makeup from the pipeline. So, any reduction in the nitrogen content from the flue gas stream is beneficial. Replacement of atmospheric air with synthetic air allows a carbon dioxide and carbon monoxide rich stream with little if any nitrogen to be utilized. Therefore, the first flue gas stream in line 135 is combusted in the CO boiler 150 in the presence of synthetic air. For synthetic air, an oxygen stream in line 177 and a carbon dioxide stream in line 172 as described later in detail may be passed to the CO boiler 150. In embodiment, the oxygen stream in line 177 and the carbon dioxide stream in line 172 may be combined and passed to the CO boiler 150 together.

In accordance with the present disclosure, a fuel gas stream in line 143 may be passed to the CO boiler 150 to provide fuel for burning the first flue gas stream in line 135. The fuel gas stream in line 143 is provided to the CO boiler 150 at a small flow rate to provide fuel for the burner but also in a large enough flow rate as needed to help regulate the internal temperature of the CO boiler 150.

In an aspect, an oxygenate stream in line 142 is passed to the CO boiler 150. In an embodiment, the oxygenate stream in line 142 is a heavy oxygenate stream. In an exemplary embodiment, the oxygenate stream in line 142 comprises one or more heavy oxygenates selected from methanol, ethanol, propanols, butanols, methyl ethyl ketone (MEK), methyl isopropyl ketone (MIPK), acetone, methanol acetate, acetic acid, formic acid, cyclohexanol, cyclopentanol, heavier alcohols and acids. Heavy oxygenates also include fusel oil. The fuel gas stream in line 143 must be available in the event the flow of the heavy oxygenates stream in line 142 to the CO boiler 150 is stopped. In accordance with the present disclosure, the heavy oxygenate stream in line 142 may be recovered from a MTO reactor effluent stream.

In an embodiment, one or more waste streams may optionally also be combusted in the CO boiler 150. A waste stream in line 144 is passed to the CO boiler 150. In an exemplary embodiment, the waste stream in line 144 comprises one or more of a diesel stream, a naphtha stream, a fuel gas stream, a lube oil stream, a skimmed heavy hydrocarbon stream, an oxygenate stream, and a fusel oil stream.

The CO boiler 150 completely burns the carbon monoxide present in the first flue gas stream to produce the carbon dioxide rich flue gas stream in line 152 having no carbon monoxide or a smaller amount of carbon monoxide compared to the amount of carbon monoxide present in the first flue gas stream in line 132. In an embodiment, the carbon dioxide rich flue gas stream in line 152 comprises oxygen from about 1 mol % to about 5 mol %. In an aspect, the carbon dioxide rich flue gas stream in line 152 comprises a higher concentration of oxygen compared to the second flue gas stream in line 133.

The carbon dioxide rich flue gas stream in line 152 may be cooled in a cooler 153 and compressed in a flue gas recycle compressor 154. Perhaps, the carbon dioxide rich flue gas stream in line 152 may be cooled in a cooler 153 and passed to a knockout drum (KOD) (not shown). From the KOD, a cooled carbon dioxide rich flue gas stream in line 152 is passed to the flue gas recycle compressor 154. In an aspect, the flue gas recycle compressor 154 is a single stage compressor. The outlet pressure of the flue gas recycle compressor 154 may range from about 207 kPa(g) (30 psig) to about 483 kPa(g) (70 psig) to meet the pressure requirement of the downstream processing.

A compressed carbon dioxide rich flue gas stream is taken from the flue gas recycle compressor 154 in line 155. In an embodiment, the compressed carbon dioxide rich flue gas stream may be separated into a carbon dioxide recycle stream in line 156 and a carbon dioxide feed stream in line 157. In accordance with the present disclosure, the carbon dioxide recycle stream in line 156 may be recycled to the CO boiler 150 or to the regenerator 120 or both. In an exemplary embodiment, the carbon dioxide recycle stream in line 156 may be recycled to both the CO boiler 150 and the regenerator 120.

In an aspect, the carbon dioxide recycle stream in line 156 may be separated into a fluidization combustion stream in line 166 and a regenerator combustion stream in line 168. The fluidization combustion stream in line 166 may also be split into a boiler combustion stream in line 169 and a lift and fluffing stream in line 173. The boiler combustion stream in line 169 taken from the fluidization combustion stream in line 166 is recycled to the CO boiler 150. In an embodiment, an optional makeup carbon dioxide stream in line 167 may be passed to the CO boiler 150. The makeup carbon dioxide stream in line 167 may be combined with the boiler combustion stream in line 169 and a combined carbon dioxide stream in line 172 may be passed to the CO boiler 150. The CO boiler 150 operates at about 0 kPa(g) (0 psig) to about 35 kPa(g) (5 psig), so the combined carbon dioxide stream in line 172 may be stepped down in pressure at some point to suit the pressure of the CO boiler.

For the CO boiler, the boiler combustion stream in line 169 is supplemented or blended with a pure oxygen stream in line 177 to create a synthetic air stream which is passed to the CO boiler 150. In an embodiment, the synthetic air stream to the CO boiler 150 may comprise about 10 mol % to about 30 mol % oxygen and the balance inert flue gas components. The non-reactive, inert portion of the stream will help moderate the internal temperature of the CO boiler 150 by removing excessive heat.

The regenerator combustion stream in line 168 may be recycled to the regenerator 120. The regenerator combustion stream in line 168 may be combined with a carbon dioxide startup stream in line 105 to provide the carbon dioxide rich oxidation stream in line 106. Also, an oxygen stream in line 104 may be combined with the regenerator combustion stream in line 168 to provide the carbon dioxide rich oxidation stream in line 106 which is passed to the regenerator 120 to burn coke from the spent catalyst.

In an embodiment, a regenerated catalyst stream may be taken in line 107 from the regenerator 120. The regenerated catalyst stream in line 107 is passed to the MTO reactor 190. A nitrogen stream in line 103 may be passed to the regenerator, particularly to the catalyst stripper 125 of the regenerator. Nitrogen may be used to strip the regenerated catalyst in the catalyst stripper 125. The nitrogen may displace the carbon oxides in the regenerated catalyst before the regenerated catalyst is transferred to the MTO reactor 190 stream in line 107.

Referring back to the fluidization combustion stream in line 166, a lift and fluffing stream may be taken in line 173 from the fluidization combustion stream. The lift and fluffing stream in line 173 may be passed to the regenerator 120. The lift and fluffing stream requires a higher pressure than the carbon dioxide rich oxidation stream in line 106 fed to the regenerator 120 and the carbon dioxide stream in line 172 fed to the CO boiler 150 pressurized by the flue gas recycle compressor 154. The lift and fluffing stream may be compressed before entering the regenerator 120. In an aspect, the lift and fluffing stream in line 173 may be cooled in a cooler 174 and compressed in a lift and fluffing gas booster compressor 175. Perhaps, the lift and fluffing stream in line 173 may be cooled in a cooler 174 and passed to a knockout drum (KOD) (not shown). From the KOD, a cooled lift and fluffing stream is passed to the lift and fluffing gas booster compressor 175. In an aspect, the lift and fluffing gas booster compressor 175 is a single stage compressor. A compressed, lift and fluffing stream is taken in line 176 from the compressor 175 and passed to the regenerator 120. The outlet pressure of the lift and fluffing gas booster compressor 175 may range from about 413 kPa(g) (60 psig) to about 758 kPa(g) (110 psig), preferably about 483 kPa(g) (70 psig) to about 689 kPa(g) (100 psig) to lift the spent catalyst charge stream in line 108 to the MTO regenerator and fluff catalyst in the MTO catalyst cooler(s) 124. Although only one catalyst cooler 124 is shown, a plurality of catalyst coolers may be employed.

In an embodiment, a portion of the compressed lift and fluffing stream in line 176 may be taken as a fluidization carbon dioxide stream in line 178 before passing to the regenerator 120. The fluidization carbon dioxide stream in line 178 is fed to the spent catalyst stream in line 102 to provide the spent catalyst charge stream in line 108. The fluidization carbon dioxide stream in line 178 fluidizes the spent catalyst as the spent catalyst charge stream in line 108 is passed to the regenerator 120. The rest of the compressed, fluffing carbon dioxide stream in line 176 is taken in line 177 and passed to the regenerator 120. In an embodiment, the compressed, fluffing carbon dioxide stream in line 177 is passed to a catalyst cooler(s) 124 of the regenerator 120 for fluffing catalyst therein.

In accordance with the present disclosure, the second flue gas stream in line 133 may be taken as a feed or to supplement a feed to the oxygenate production unit 180 of the MTO unit 111. In an embodiment, the second flue gas stream in line 133 may be combined with the carbon dioxide feed stream in line 157 to provide a carbon oxide feed stream in line 161. The carbon oxide feed stream in line 161 is passed to the oxygenate production unit 180.

The carbon oxide feed stream in line 161 may be cooled in a cooler 162 and compressed in a carbon dioxide product compressor 163. Perhaps, the carbon oxide feed stream in line 161 may be cooled in a cooler 162 and passed to a knockout drum (KOD) (not shown). From the KOD, a cooled carbon oxide feed stream is passed to the carbon dioxide product compressor 163. In an aspect, the carbon dioxide product compressor 163 is a multistage compressor. In an exemplary embodiment, the carbon oxide feed stream in line 161 may be compressed to a pressure of about 1.5 MPa(g) (218 psig) to about 4.5 MPa(g) (653 psig) in the carbon dioxide product compressor 163. A compressed carbon oxide feed stream is taken in line 164 from the carbon dioxide product compressor 163 and passed to the oxygenate production unit 180 to produce methanol from the carbon oxides. The methanol from the oxygenate production unit 180 may be converted to olefins in the MTO reactor 190.

The combined carbon dioxide stream in line 172 is a recycle stream taken from the carbon dioxide rich flue gas stream in line 152, so it reduces carbon dioxide that would be needed to be made up in line 167 to the CO boiler 150 in line 172 and to the MTO regenerator 120 in line 105. If fresh carbon dioxide is made up at both the CO boiler 150 in line 167 and the MTO regenerator 120 in line 105, this carbon dioxide effectively passes through the MTO unit and is sent to the oxygenate production unit 180. Carbon dioxide must be compressed to a pressure of about 1.5 MPa(g) (218 psig) to about 4.5 MPa(g) (653 psig) in the carbon dioxide product compressor 163 to enter the oxygenate production unit 180. Carbon dioxide made up to MTO unit to make synthetic air will pass through the MTO unit and will have to be compressed from low pressure of about 0 kPa(g) (0 psig) to about 345 kPa(g) (50 psig) to about 1.5 MPa(g) (218 psig) to about 4.5 MPa(g) (653 psig) at substantial expense to enter the oxygenate production unit 180. Reducing the make-up carbon dioxide to the MTO unit and only compressing necessary make up carbon dioxide in line 167 and line 105 reduces compression energy expense by sending less carbon oxide to the oxygenate production unit 180. The present process achieves this reduction by recycling low pressure CO boiler flue gas from the CO boiler 150 as lift and fluffing gas stream in line 173 to the MTO regenerator 120 or as blending gas in the combined carbon dioxide stream in line 172 with pure oxygen stream in line 177 to make the synthetic air for combustion and heat moderation in the CO boiler 150. Further, the captured carbon oxide streams in lines 133 and 157 may not be utilized in the CO boiler 150 or the regenerator 120. The streams in line 133 and 157 may be used as valuable feed streams and charged to the oxygenate production unit 180 to produce methanol.

The carbon monoxide rich second flue gas stream in line 133 is passed to the oxygenate production unit 180. Carbon monoxide is a better reactant for making methanol.

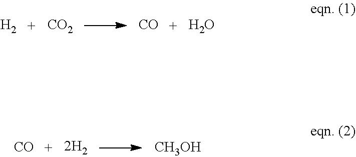

The first reaction of carbon dioxide synthesis to methanol involves reacting one mole of hydrogen with each mole of carbon dioxide to generate carbon monoxide and water. One mole of carbon monoxide therefore saves one mole of hydrogen as compared to one mole of carbon dioxide because the mole of carbon dioxide does not need to be converted to carbon monoxide.

In the CO boiler 150, carbon monoxide is oxidized to carbon dioxide. An important parameter for this reaction is the carbon monoxide to molecular oxygen ratio. Ideally this ratio would be above 2, so that each oxygen atom in the oxygen molecule introduced is reacted with a carbon monoxide molecule. Excess oxygen is undesirable for methanol synthesis because oxygen converts to water while consuming hydrogen and makes no methanol. If possible, no excess oxygen should be left in the flue gas from the CO boiler because it will require additional hydrogen to be consumed in the oxygenate production unit 180 and consumes hydrolyzing electricity to provide excess oxygen in line 177. In a typical process, the carbon monoxide to oxygen ratio is found to be less than 2. An unexpected benefit from the present process is that the carbon monoxide to molecular oxygen ratio is found to be well above 3 by recycling some of the flue gas to the regenerator 120 and the CO boiler 150 before recycling it to the oxygenate production unit 180.

In the present process, the carbon dioxide rich flue gas stream in line 152 from the CO boiler 150 contains almost no carbon monoxide or a small amount of carbon monoxide and sufficient excess oxygen is added to the CO boiler 150 in line 177 only to ensure complete combustion of the carbon monoxide to carbon dioxide. Typically, this oxygen is sent to the oxygenate production unit 180. But, in the present process this oxygen is recycled to the MTO regenerator 120 and/or the CO boiler 150. Oxygen will not build up in the system because the amount of fresh oxygen added to the regenerator in line 104 and to the CO boiler in line 177 not consumed is reduced proportionately by the amount of oxygen being returned in the carbon dioxide recycle stream in line 156 from the CO boiler 150.

Referring back to the oxygenate production unit 180, the carbon oxide feed stream in line 164 may be passed to a methanol synthesis section to produce methanol from the carbon oxides. A hydrogen stream in line 158 may also be passed to the oxygenate production unit 180. In an aspect, the hydrogen stream in line 158 may be combined with the carbon oxide feed stream in line 164 and passed to the oxygenate production unit 180.

The methanol synthesis process is accomplished in the presence of a methanol synthesis catalyst. A suitable methanol synthesis catalyst may be a copper on a zinc oxide and alumina support. Synthesis conditions for the methanol synthesis section of the oxygenate production unit 180 may include a temperature of about 200° C. to about 300° C. and a pressure of about 3.5 to about 10 MPa. Reaction equilibrium typically requires methanol separation and recycle of unreacted reagents to the synthesis reaction.

From the methanol synthesis section, an effluent stream may be passed to the methanol purification section to separate methanol from the reactor effluent. A methanol stream is taken in line 182 from the oxygenate production unit 180. The methanol stream in line 182 is passed to the MTO reactor 190.

In the MTO reactor 190, the methanol stream in line 182 is reacted with an MTO catalyst. In an embodiment, the MTO catalyst is a fluidized MTO catalyst. In an aspect, the MTO catalyst may be a silicoaluminophosphate (SAPO) catalyst. SAPO catalysts and their formulation are generally taught in U.S. Pat. Nos. 4,499,327A, 10,358,394 and 10,384,986.

The MTO reactor 190 is maintained at effective conditions for the conversion of the oxygenate comprising methanol to produce light olefin products and generate oxygenated byproducts. A hot vaporous reactor effluent stream is withdrawn from the MTO reactor 190 in line 192. The hot vaporous reactor effluent stream in line 192 may comprise light olefins, water, and oxygenates. Light olefins are separated from the hot vaporous reactor effluent stream in line 192. In an embodiment, the heavy oxygenate stream in line 142 is separated in a byproduct stream from the hot vaporous reactor effluent stream in line 192. In an embodiment, the reaction conditions of the MTO reactor 190 may include a reaction pressure between about 0.1 MPa(a) (15 psia) and about 0.7 MPa(a) (100 psia) and a reaction temperature between about 400° C. (750° F.) to about 525° C. (980° F.).

The spent catalyst stream is taken from the MTO reactor 190 in line 102 and passed to the regenerator 120 to burn coke from the catalyst. A regenerated catalyst stream is taken from the regenerator 120 in line 107 and passed to the MTO reactor 190. A periodic or continuous circulation of the fluidized catalyst is provided between the MTO reactor 190 and the regenerator 120 for example to maintain the selectivity and the conversion desired.

In another exemplary embodiment, the fluidization combustion stream in line 166 is optional. In such an embodiment, the compressed carbon dioxide rich flue gas stream in line 155 is separated into the carbon dioxide recycle stream in line 156 and the carbon dioxide feed stream in line 157. The carbon dioxide recycle stream in line 156 is recycled to the regenerator 120 in the carbon dioxide rich oxidation stream in line 106. The carbon dioxide feed stream in line 157 is passed to the oxygenate production unit 180. In this embodiment, all of the carbon dioxide rich flue gas stream in line 152 not needed for the carbon dioxide recycle stream in line 156, is taken in line 157 and passed to the oxygenate production unit 180.

Any of the above lines, conduits, units, devices, vessels, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect. Further, the figure may include one or more exemplary sensors located on one or more conduits. Nevertheless, there may be sensors present on every stream so that the corresponding parameter(s) can be controlled accordingly.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring component, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

### Example

An experimental study was performed. The carbon dioxide rich flue gas stream in line 152 was taken from the CO boiler 150. The operating conditions, and feed rate of various streams to the CO boiler 150 are as below in the Table.

TABLE

| | | | |
|---|---|---|---|
| CO Boiler Operating | Temperature, ° C. (° F.) | 982 | (1800) |
| Conditions | Pressure, kPa(g) (psig) | 3.5 | (0.5) |
| Feed Rate to the | Flue gas, Kg/hr (lb/hr) | 19505 | (43000) |
| CO Boiler | Oxygenates, Kg/hr (lb/hr) | 563 | (1240) |
| | Fuel gas, Kg/hr (lb/hr) | 222 | (490) |
| | Oxygen, Kg/hr (lb/hr) | 2110 | (4650) |
| | Carbon dioxide, Kg/hr (lb/hr) | 11608 | (25590) |

A carbon dioxide recycle stream was taken from the carbon dioxide rich flue gas stream of the CO boiler. For the CO boiler, the carbon dioxide recycle stream was blended with pure oxygen to create a synthetic air stream that comprises about 20% mol oxygen and the balance inert flue gas components. The non-reactive 80% of the stream will help moderate the internal temperature of the CO boiler by removing excessive heat. The observations and the results of the study are as below:

### Lift and Fluffing Gas to the Regenerator

The lift and fluffing stream for the regenerator was taken from the carbon dioxide recycle stream. The lift and fluffing stream required compression of the carbon dioxide recycle stream from 0 kPa(g) (0 psig) to 69 kPa(g) (10 psig) to about 621 kPa(g) (90 psig) to enter the regenerator lift riser and regenerator catalyst coolers. Taking carbon dioxide for lift and fluffing in the MTO reactor would reduce the flow rate of carbon dioxide compressed for recycle to the oxygenate production unit. For the reduced compression, the size of the compressor needed was also decreased. CO₂ compressor capital cost savings was estimated at $410,000 and the estimated CO₂ compressor power savings was 0.73 MW.

### Oxygen-Blending Gas to the CO Boiler

The pressure requirement was much lower since the CO boiler operates at about 13.8 kPa(g) (2 psig) and would likewise reduce the flow rate of carbon dioxide compressed. Since this carbon dioxide is recycled to the CO boiler, it reduces the flow rate of carbon dioxide that would be required to be made up and would reduce the amount of carbon dioxide that would need to be compressed to about 3103 kPa(g) (450 psig) to enter the oxygenate production unit, lowering the total carbon dioxide recycle compressor cost of the MTO flue gas system. For the reduced compression requirement, CO₂ compressor capital cost savings was estimated at $330,000 and the CO₂ compressor estimated power savings was 0.6 MW.

### Specific Embodiments

While the following is described in conjunction with specific embodiments, it will be understood that this description is intended to illustrate and not limit the scope of the preceding description and the appended claims.

A first embodiment of the present disclosure is a process of separating carbon oxides from a flue gas stream, comprising separating a flue gas stream into a first flue gas stream and a second flue gas stream; combusting the first flue gas stream with an oxygen stream in a boiler to provide a carbon dioxide rich flue gas stream; taking a carbon dioxide recycle stream from the carbon dioxide rich flue gas stream; and passing the second flue gas stream to an oxygenate production unit, and recycling the carbon dioxide recycle stream to the boiler or to a regenerator or both. An embodiment of the present disclosure is one, any or all of prior embodiments in this paragraph up through the first embodiment in this paragraph wherein, the carbon dioxide rich flue gas stream comprises a higher oxygen concentration than the second flue gas stream. An embodiment of the present disclosure is one, any or all of prior embodiments in this paragraph up through the first embodiment in this paragraph further comprising compressing the carbon dioxide rich flue gas stream to provide a compressed carbon dioxide rich flue gas stream; and taking the carbon dioxide recycle stream from the compressed carbon dioxide rich flue gas stream. An embodiment of the present disclosure is one, any or all of prior embodiments in this paragraph up through the first embodiment in this paragraph further comprising combusting carbon monoxide in the first flue gas stream to carbon dioxide in the boiler to provide the carbon dioxide rich flue gas stream. An embodiment of the present disclosure is one, any or all of prior embodiments in this paragraph up through the first embodiment in this paragraph further comprising taking a carbon dioxide feed stream from the carbon dioxide rich flue gas stream; mixing the carbon dioxide feed stream with the second flue gas stream to provide a carbon oxide feed stream; and converting the carbon oxide feed stream into methanol in the oxygenate production unit. An embodiment of the present disclosure is one, any or all of prior embodiments in this paragraph up through the first embodiment in this paragraph further comprising compressing the carbon oxide rich stream to provide a compressed carbon oxide stream; and passing the compressed carbon oxide stream to the oxygenate production unit. An embodiment of the present disclosure is one, any or all of prior embodiments in this paragraph up through the first embodiment in this paragraph further comprising taking a combustion carbon dioxide stream from the carbon dioxide recycle stream; mixing the combustion carbon dioxide stream with an oxygen stream to provide a carbon dioxide rich oxidation stream; and passing the carbon dioxide rich oxidation stream to the regenerator to burn coke from a spent catalyst and provide the flue gas stream. An embodiment of the present disclosure is one, any or all of prior embodiments in this paragraph up through the first embodiment in this paragraph further comprising taking a gas stream from the carbon dioxide recycle stream and passing the gas stream to the regenerator. An embodiment of the present disclosure is one, any or all of prior embodiments in this paragraph up through the first embodiment in this paragraph further comprising taking a catalyst cooler fluffing gas stream and/or a lift gas stream from the carbon dioxide recycle stream and passing the catalyst cooler fluffing gas and/or the lift gas stream to the regenerator. An embodiment of the present disclosure is one, any or all of prior embodiments in this paragraph up through the first embodiment in this paragraph further comprising taking a boiler combustion carbon dioxide stream from the carbon dioxide recycle stream; passing the boiler combustion carbon dioxide stream, a boiler combustion oxygen stream, and an oxygenate stream to the boiler; and combusting the oxygenate stream and the first flue gas stream with the boiler oxygen stream in the boiler to provide the carbon dioxide rich flue gas stream. An embodiment of the present disclosure is one, any or all of prior embodiments in this paragraph up through the first embodiment in this paragraph further comprising combusting a waste stream in the boiler. An embodiment of the present disclosure is one, any or all of prior embodiments in this paragraph up through the first embodiment in this paragraph wherein, the waste stream comprises one or more of a diesel stream, a naphtha stream, a fuel gas stream, a lube oil stream, a skimmed heavy hydrocarbon stream, an oxygenate stream, and a fusel oil stream. An embodiment of the present disclosure is one, any or all of prior embodiments in this paragraph up through the first embodiment in this paragraph wherein the oxygenate stream is taken from the oxygenate production unit. An embodiment of the present disclosure is one, any or all of prior embodiments in this paragraph up through the first embodiment in this paragraph wherein the oxygenate stream comprises one or more heavy oxygenates selected from methanol, ethanol, propanol, butanol, methyl ethyl ketone (MEK), methyl isopropyl ketone (MIPK), acetone, methanol acetate, acetic acid, formic acid, cyclohexanol, cyclopentanol, heavier alcohols and acids.

A second embodiment of the present disclosure is a process of separating carbon oxides from a flue gas stream, comprising separating a flue gas stream into a first flue gas stream and a second flue gas stream; combusting the first flue gas stream with an oxygen stream in a boiler to combust carbon monoxide to carbon dioxide and provide a carbon dioxide rich flue gas stream; compressing the carbon dioxide rich flue gas stream to provide a compressed carbon dioxide rich flue gas stream; taking a carbon dioxide recycle stream from the compressed carbon dioxide rich flue gas stream; and passing the second flue gas stream to an oxygenate production unit; and recycling the carbon dioxide recycle stream to the boiler or to a regenerator or both. An embodiment of the present disclosure is one, any or all of prior embodiments in this paragraph up through the second embodiment in this paragraph further comprising taking a boiler combustion carbon dioxide stream from the carbon dioxide recycle stream; passing the boiler combustion carbon dioxide stream, a boiler combustion oxygen stream, and an oxygenate stream to the boiler; and combusting the oxygenate stream and the first flue gas stream with the boiler oxygen stream in the boiler to provide the carbon dioxide rich flue gas stream. An embodiment of the present disclosure is one, any or all of prior embodiments in this paragraph up through the second embodiment in this paragraph wherein, the carbon dioxide rich flue gas stream comprises a higher oxygen concentration than the second flue gas stream. An embodiment of the present disclosure is one, any or all of prior embodiments in this paragraph up through the second embodiment in this paragraph further comprising taking a carbon dioxide feed stream from the compressed carbon dioxide rich flue gas stream; mixing the carbon dioxide feed stream with the second flue gas stream to provide a carbon oxide feed stream; and converting the carbon oxide feed stream into methanol in the oxygenate production unit.

A third embodiment of the present disclosure is a process of separating carbon oxides from a flue gas stream, comprising separating a flue gas stream into a first flue gas stream and a second flue gas stream; combusting the first flue gas stream with an oxygen stream in a boiler to combust carbon monoxide to carbon dioxide and provide a carbon dioxide rich flue gas stream; compressing the carbon dioxide rich flue gas stream to provide a compressed carbon dioxide rich flue gas stream; taking a lift gas stream from the compressed carbon dioxide rich flue gas stream; and passing the lift gas stream and a carbon dioxide rich oxidation stream to the regenerator to burn coke from a spent catalyst and provide the flue gas stream. An embodiment of the present disclosure is one, any or all of prior embodiments in this paragraph up through the third embodiment in this paragraph further comprising taking a combustion carbon dioxide stream from the compressed carbon dioxide rich flue gas stream; and mixing the combustion carbon dioxide stream with an oxygen stream to provide the carbon dioxide rich oxidation stream. An embodiment of the present disclosure is one, any or all of prior embodiments in this paragraph up through the third embodiment in this paragraph further comprising taking a boiler combustion carbon dioxide stream from compressed carbon dioxide rich flue gas stream; passing the boiler combustion carbon dioxide stream, a boiler combustion oxygen stream, and an oxygenate stream to the boiler; and combusting the oxygenate stream and the first flue gas stream with the boiler oxygen stream in the boiler to provide the carbon dioxide rich flue gas stream.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of separating carbon oxides from a flue gas stream, comprising
separating a flue gas stream into a first flue gas stream and a second flue gas stream;
combusting said first flue gas stream with an oxygen stream in a boiler to provide a carbon dioxide rich flue gas stream;
taking a carbon dioxide recycle stream from said carbon dioxide rich flue gas stream; and
passing said second flue gas stream to an oxygenate production unit; and
recycling said carbon dioxide recycle stream to the boiler or to a regenerator or both.

2. The process of claim 1 wherein, said carbon dioxide rich flue gas stream comprises a higher oxygen concentration than said second flue gas stream.

3. The process of claim 1 further comprising:
compressing said carbon dioxide rich flue gas stream to provide a compressed carbon dioxide rich flue gas stream; and
taking said carbon dioxide recycle stream from said compressed carbon dioxide rich flue gas stream.

4. The process of claim 1 further comprising combusting carbon monoxide in said first flue gas stream to carbon dioxide in the boiler to provide said carbon dioxide rich flue gas stream.

5. The process of claim 1 further comprising:
taking a carbon dioxide feed stream from said carbon dioxide rich flue gas stream;
mixing said carbon dioxide feed stream with said second flue gas stream to provide a carbon oxide feed stream; and
converting said carbon oxide feed stream into methanol in the oxygenate production unit.

6. The process of claim 5 further comprising:
compressing said carbon oxide rich stream to provide a compressed carbon oxide stream; and
passing said compressed carbon oxide stream to the oxygenate production unit.

7. The process of claim 3 further comprising:
taking a combustion stream from said carbon dioxide recycle stream;
mixing said combustion stream with an oxygen stream to provide a carbon dioxide rich oxidation stream; and
passing said carbon dioxide rich oxidation stream to the regenerator to burn coke from a spent catalyst and provide said flue gas stream.

8. The process of claim 7 further comprising taking a lift gas stream from said carbon dioxide recycle stream and passing said lift gas stream to the regenerator.

9. The process of claim 7 further comprising taking a catalyst cooler fluidization gas stream from said carbon dioxide recycle stream and passing the said catalyst cooler fluidization gas to the catalyst cooler of the regenerator.

10. The process of claim 3 further comprising:
taking a boiler combustion stream from said carbon dioxide recycle stream;
passing said boiler combustion stream, a boiler combustion oxygen stream, and an oxygenate stream to the boiler; and
combusting said oxygenate stream and said first flue gas stream with said boiler oxygen stream in the boiler to provide said carbon dioxide rich flue gas stream.

11. The process of claim 7 further comprising combusting a waste stream in the boiler.

12. The process of claim 11, wherein said waste stream comprises one or more of a diesel stream, a naphtha stream, a fuel gas stream, a lube oil stream, a skimmed heavy hydrocarbon stream, an oxygenate stream, and a fusel oil stream.

13. The process of claim 10 wherein said oxygenate stream is taken from the oxygenate conversion unit.

14. The process of claim 13 wherein said oxygenate stream comprises one or more heavy oxygenates selected from methanol, ethanol, propanol, butanol, methyl ethyl ketone, methyl isopropyl ketone, acetone, methanol acetate, acetic acid, formic acid, cyclohexanol, and cyclopentanol.

15. A process of separating carbon oxides from a flue gas stream, comprising
separating a flue gas stream into a first flue gas stream and a second flue gas stream;
combusting said first flue gas stream with an oxygen stream in a boiler to combust carbon monoxide to carbon dioxide and provide a carbon dioxide rich flue gas stream;
compressing said carbon dioxide rich flue gas stream to provide a compressed carbon dioxide rich flue gas stream;
taking a carbon dioxide recycle stream from said compressed carbon dioxide rich flue gas stream;
passing said second flue gas stream to an oxygenate production unit; and
recycling said carbon dioxide recycle stream to the boiler or to a regenerator or both.

16. The process of claim 15 further comprising:
taking a boiler combustion stream from said carbon dioxide recycle stream;
passing said boiler combustion stream, a boiler combustion oxygen stream, and an oxygenate stream to the boiler; and
combusting said oxygenate stream and said first flue gas stream with said boiler oxygen stream in the boiler to provide said carbon dioxide rich flue gas stream.

17. The process of claim 15 wherein, said carbon dioxide rich flue gas stream comprises a higher oxygen concentration than said second flue gas stream.

18. The process of claim 15 further comprising:
taking a carbon dioxide feed stream from said compressed carbon dioxide rich flue gas stream;
mixing said carbon dioxide feed stream with said second flue gas stream to provide a carbon oxide feed stream; and
converting said carbon oxide feed stream into methanol in the oxygenate production unit.

19. A process of separating carbon oxides from a flue gas stream, comprising
separating a flue gas stream into a first flue gas stream and a second flue gas stream;
combusting said first flue gas stream with an oxygen stream in a boiler to combust carbon monoxide to carbon dioxide and provide a carbon dioxide rich flue gas stream;
compressing said carbon dioxide rich flue gas stream to provide a compressed carbon dioxide rich flue gas stream;
taking a lift gas stream from said compressed carbon dioxide rich flue gas stream; and
passing said lift gas stream and a carbon dioxide rich oxidation stream to the regenerator to burn coke from a spent catalyst and provide said flue gas stream.

20. The process of claim 19 further comprising:
taking a combustion stream from said compressed carbon dioxide rich flue gas stream; and
mixing said combustion stream with an oxygen stream to provide said carbon dioxide rich oxidation stream.

21. The process of claim 19 further comprising:
taking a boiler combustion stream from compressed carbon dioxide rich flue gas stream;
passing said boiler combustion stream, a boiler combustion oxygen stream, and an oxygenate stream to the boiler; and
combusting said oxygenate stream and said first flue gas stream with said boiler oxygen stream in the boiler to provide said carbon dioxide rich flue gas stream.
